(19) United States
(12) Patent Application Publication
(10) Pub. No.: US 2022/0023455 A1
(43) Pub. Date: Jan. 27, 2022

(54) PORTABLE SANITIZATION SYSTEM AND METHOD WITH ULTRAVIOLET AND VISIBLE LIGHT SENSING

(71) Applicant: HONEYWELL INTERNATIONAL INC., Morris Plains, NJ (US)

(72) Inventors: Stephen Yates, Des Plaines, IL (US); Peter Michalakos, Des Plaines, IL (US)

(73) Assignee: HONEYWELL INTERNATIONAL INC., Morris Plains, NJ (US)

(21) Appl. No.: 16/937,182

(22) Filed: Jul. 23, 2020

(51) Int. Cl. A61L 2/10; A61L 2/28; A61L 2/24

(52) U.S. Cl. CPC A61L 2/10; A61L 2/28; A61L 2202/25; A61L 2202/11; A61L 2202/16; A61L 2/24

(57) ABSTRACT

A system and method for sanitizing a surface within an aircraft includes emitting UV radiation from a UV radiation source toward the surface, and emitting visible light from a visible light source toward the surface. A UV sensor senses an intensity of the UV radiation reflected by a reflector that is disposed on a least a portion of the surface and a visible light senses an intensity of the visible light reflected by the reflector. A processor processes the UV intensity signal and the visible light intensity signal to determine the intensity of the UV radiation on the surface and a UV dose to the surface.

### TECHNICAL FIELD

The present disclosure generally relates to sanitizing systems, and more particularly relates to portable sanitization systems and methods for aircraft cabin surfaces.

### BACKGROUND

Infectious disease transmission among air travelers has been, and continues to be, both a personal and public health concern. Numerous and varied viruses, bacteria, and fungal pathogens can be spread through the air and via contact surfaces. Thus, many private and commercial airliners use extensive on-board air filtration and, in some instances, ultraviolet (UV) technologies in areas outside the cabin compartment to reduce the spread of various airborne. Nonetheless, disease transmission continues, suggesting cabin surfaces may be contributing factors to such transmission.

Chemical disinfection of surfaces is labor intensive and may leave potentially undesirable residues. It is known that UV radiation, and especially UVC radiation, can be used to disinfect air, water, and surfaces, to thereby mitigate the risk of infectious disease transmission. Thus, various devices and systems have been developed that use UV radiation to sanitize passenger aircraft interior surfaces. One such device comprises a cart that can pushed down the aisle of an aircraft. The cart is equipped with arms that can extend over the seats. Ultraviolet lamps on the arms are used to emit UVC radiation and sanitize the seats and other touch surfaces in the cabin. While this device has been shown to be effective, it does suffer certain drawbacks. For example, it may not provide feedback to the user and/or customer as to whether the sanitization has been done correctly and thoroughly.

Hence, there is a need for portable sanitization system that uses UV radiation to sanitize aircraft cabin surfaces and that provides real-time feedback as to whether the sanitization has been done correctly and thoroughly. The present invention addresses at least this need.

### BRIEF SUMMARY

This summary is provided to describe select concepts in a simplified form that are further described in the Detailed Description. This summary is not intended to identify key or essential features of the claimed subject matter, nor is it intended to be used as an aid in determining the scope of the claimed subject matter.

In one embodiment, a portable sanitization system for sanitizing a surface in an aircraft includes a mobile cart, an arm, an ultraviolet (UV) radiation source, a visible light source, a reflector, a UV sensor, a visible light source, and a processor. The mobile cart is movable within the aircraft. The arm is coupled to, and is movable relative to, the mobile cart. The UV radiation source is mounted on the arm and is operable to emit UV radiation toward the surface. The visible light source is mounted on the arm and is operable to emit visible light toward the surface. The reflector is disposed on at least a portion of the surface and is configured to reflect at least a portion of the UV radiation and at least a portion of the visible light emitted toward the surface. The UV sensor is mounted on the mobile cart and is shielded from the UV radiation source. The UV sensor is operable to (i) sense an intensity of the UV radiation reflected by the reflector and (ii) supply a UV intensity signal representative thereof. The visible light sensor is mounted on the mobile cart and is shielded from the UV radiation source. The visible light sensor is operable to (i) sense an intensity of the visible light reflected by the reflector and (ii) supply a visible light intensity signal representative thereof. The processor is coupled to receive the UV intensity signal from the UV sensor and the visible light intensity signal from the visible light sensor. The processor is configured to determine (i) the intensity of the UV radiation on the surface based on the UV intensity signal and the visible light intensity signal and (ii) a UV dose to the surface based at least in part on the determined intensity of the UV radiation.

In another embodiment, a portable sanitization system for sanitizing surfaces in an aircraft includes a mobile cart, a plurality of arms, a plurality of ultraviolet (UV) radiation sources, a visible light source, a plurality of reflectors, a plurality of UV sensors, a plurality of visible light sensors, a processor, a wireless transmitter, and a receiver. The mobile cart is movable within the aircraft, the arms are coupled to, and extendable from, the mobile cart. Each UV radiation source is mounted on a different one of the arms and is operable to emit UV radiation toward the surfaces. The visible light source is mounted on the arm and is operable to emit visible light toward the surfaces. Each reflector is disposed on at least a portion of a different one of the surfaces and is configured to reflect at least a portion of the UV radiation and at least a portion of the visible light emitted toward the surfaces. Each UV sensor is mounted on a different one of the arms and is shielded from the UV radiation sources. Each UV sensor is operable to (i) sense an intensity of the UV radiation reflected by each reflector and (ii) supply a UV intensity signal representative thereof. Each visible light sensor is mounted on a different one of the arms and is shielded from the UV radiation source. Each visible light sensor is operable to (i) sense an intensity of the visible light reflected by each reflector and (ii) supply a visible light intensity signal representative thereof. The processor is coupled to receive the UV intensity signals from the UV sensors and the visible light intensity signals from the visible light sensors. The processor is configured to (i) pulse each visible light source on and off at a predetermined frequency, (ii) determine the intensity of the UV radiation on each surface based on the UV intensity signals and the visible light intensity signals, and (iii) determine a UV dose to each surface based at least in part on the determined intensity of the UV radiation. The wireless transmitter is in operable communication with the processor and is responsive to commands from the processor to wirelessly transmit at least data representative of the UV dose to each surface. The receiver is in operable communication with the wireless transmitter to receive the data transmitted therefrom.

In yet another embodiment, a method of sanitizing a surface within an aircraft using a mobile cart within the aircraft, where the mobile cart has an arm that is coupled thereto and that is movable relative thereto, the arm has an ultraviolet (UV) radiation source and a visible light source mounted thereon, and a UV sensor and a visible light source are mounted on the mobile cart. The method includes emitting UV radiation from the UV radiation source toward the surface; emitting visible light from the visible light source toward the surface; sensing, using the UV sensor, an intensity of the UV radiation reflected by a reflector that is disposed on a least a portion of the surface; supplying a UV intensity signal from the UV sensor to a processor, the UV intensity signal representative of the sensed intensity of the UV radiation reflected by the reflector; sensing, using the visible light sensor, an intensity of the visible light reflected by the reflector; supplying a visible light intensity signal from the visible light sensor to the processor, the visible light intensity signal representative of the sensed intensity of the visible light reflected by the reflector; and processing, in the processor, the UV intensity signal and the visible light intensity signal to determine (i) the intensity of the UV radiation on the surface and (ii) a UV dose to the surface.

Furthermore, other desirable features and characteristics of the portable sanitization system and method will become apparent from the subsequent detailed description and the appended claims, taken in conjunction with the accompanying drawings and the preceding background.

### BRIEF DESCRIPTION OF THE DRAWINGS

The present invention will hereinafter be described in conjunction with the following drawing figures, wherein like numerals denote like elements, and wherein:

FIG. 1 depicts a simplified schematic representation of one embodiment of a portable sanitization system;

FIG. 2 depicts a simplified schematic representation of another embodiment of a portable sanitization system;

FIG. 3 depicts a representation of the portable sanitization system of FIG. 2 moving through the aisle of an aircraft cabin; and

FIG. 4 depicts a process, which may be implemented by the portable sanitization systems of FIGS. 1 and 2, to sanitize a surface within an aircraft.

### DETAILED DESCRIPTION

The following detailed description is merely exemplary in nature and is not intended to limit the invention or the application and uses of the invention. As used herein, the word “exemplary” means “serving as an example, instance, or illustration.” Thus, any embodiment described herein as “exemplary” is not necessarily to be construed as preferred or advantageous over other embodiments. All of the embodiments described herein are exemplary embodiments provided to enable persons skilled in the art to make or use the invention and not to limit the scope of the invention which is defined by the claims. Furthermore, there is no intention to be bound by any expressed or implied theory presented in the preceding technical field, background, brief summary, or the following detailed description.

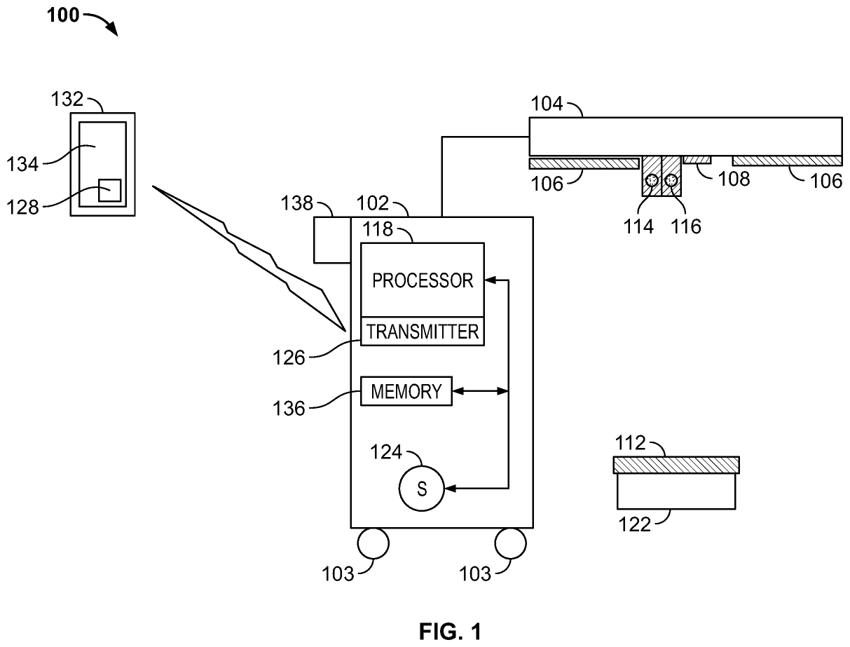

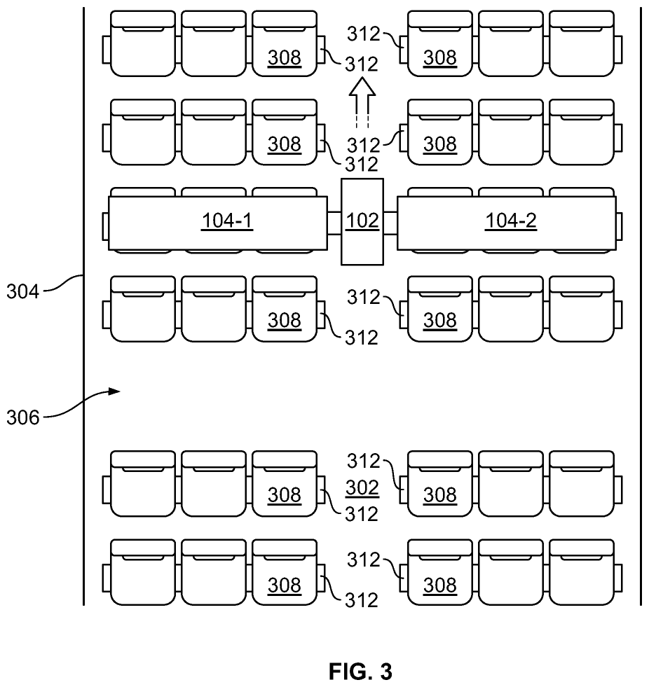

Referring to FIG. 1, a simplified schematic representation of one embodiment of a portable sanitization system 100 is depicted. The depicted system 100 includes a mobile cart 102, an arm 104, an ultraviolet radiation source 106, a visible light source 108, a reflector 112, a UV sensor 114, a visible light sensor 116, and a processor 118. The mobile cart 102 is movable within an aircraft. In particular, it includes a plurality of wheels 103 and, as depicted in FIG. 3, is dimensioned to be movable through the aisle 302 of an aircraft 304.

The arm 104 is coupled to, and is movable relative to, the mobile cart 102. The arm 104 may thus extend out from the mobile cart 102 and over various surfaces 122 in the aircraft cabin 306; for example, over the seats 308 (see FIG. 3). The arm 104 may be movable relative to the mobile cart 102 using any one of numerous techniques. For example, it may rotate about an axis relative to the cart 102, it may extend in an articulating manner from the cart 102, it may extend in a telescoping manner from the cart 102, or it may extend in scissor-like manner from the cart 102, just to name a few non-limiting techniques.

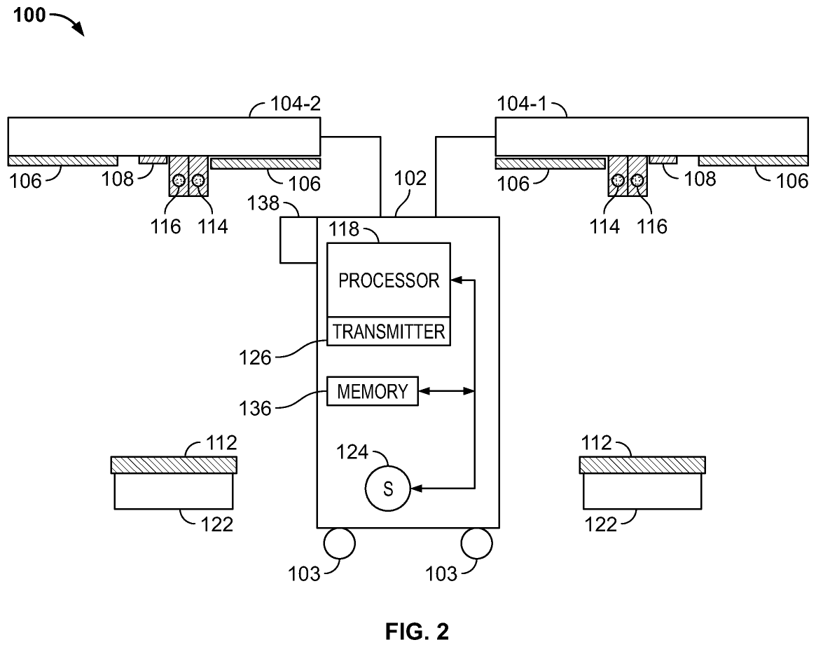

It should be noted that although the system 100 depicted in FIG. 1 includes one arm 104, the system 100 may also be equipped with a plurality of arms 104. An example of one such embodiment is depicted in FIG. 2, which includes two arms 104-1, 104-2. It will be appreciated, however, that the system 100 could include more than two arms 104 (e.g., 104-1, 104-2, 104-3, . . . 104-N).

Regardless of the number of arms 104, one or more UV radiation sources 106 are mounted on each arm 104 and each UV radiation source 106 is operable to emit UV radiation toward the surface 122. Each UV radiation source 106 may be variously implemented. For example, each UV radiation source 106 may be implemented using fluorescent lamps, light emitting diodes (LEDs), mercury vapor lamps, pulsed-Xenon and various other UV-emitting devices, just to name a few. In one particular embodiment, each UV radiation source 106 is implemented using mercury vapor lamps. Although the UV radiation wavelength emitted by each UV radiation source 106 may vary, in a particular preferred embodiment, each UV radiation source 106 is configured to emit UV radiation having wavelengths in the UV-C band (e.g., 200 nm to 280 nm).

A visible light source 108 is also mounted on each arm 104 and each visible light source 108 is operable to emit visible light toward the surface 122. Each visible light source 108 may also be variously implemented. For example, each visible light source 108 may be implemented using fluorescent lamps, light emitting diodes (LEDs), incandescent lamps, just to name a few. Although the wavelength emitted by each visible light source may vary, in a particular preferred embodiment, each visible light source 108 is configured to emit light that is different from visible white light. For example, light having wavelengths of green, red, or blue light so that it is distinguishable from the ambient light in the aircraft cabin 306.

A reflector 112 is disposed on at least a portion of each surface 122. Each reflector 112 is configured to reflect at least a portion of the UV radiation and at least a portion of the visible light emitted toward the surface 122 on which the reflector 112 is disposed. The reflectors 112 may be variously formed and implemented. For example, the reflectors 112 may be formed integrally with the surfaces 122, may be readily disposable on, and removable from, the surfaces 122, or both. The reflectors 112 that are formed integrally with the surfaces 122 may, for example, be installed as decorative features in portions of the seats 308 and/or armrests 312, or may be part of one or more aircraft surfaces that are sufficiently reflective without any modification. The reflectors 112 may be made of various materials. Some non-limiting examples include polished aluminum and porous polytetrafluoroethylene (PTFE), just to name a few. The reflectors 112 may also be either specular or diffuse. Specular reflectors reflect light incident on them at a single angle relative to the angle of incidence, such as a mirror. Diffuse reflectors distribute the light at many angles, like a projector screen or reflectors used on highway signs. In a preferred embodiment, diffuse reflectors are used so that the angle at which the reflectors 112 are positioned relative to the UV and visible light sources is less important.

A UV sensor 114 is mounted on the mobile cart 102, and each is shielded from the UV radiation sources 106. More specifically, each UV sensor 114 is shielded from the UV radiation emitted directly (e.g., not reflected) from the radiation sources 106, and receives only reflected UV radiation. The UV sensors 114 are each operable to sense the intensity of the UV radiation reflected by the reflectors 112. The UV sensors 114 are also each operable to supply (via either wired or wireless techniques) a UV intensity signal representative of the sensed intensity to the processor 118. The UV sensors 114 may be implemented using any one of numerous known UV sensors now known or developed in the future. Some non-limiting examples of suitable UV sensors 114 include various charge-coupled devices (CCD), phototubes, and photodiodes, just to name a few. In the depicted embodiment, a UV sensor 114 is mounted on each arm 104. In other embodiments, one or more UV sensors 114 may be mounted on the mobile cart 102, remote from each arm 104. In this embodiment, optical fibers may be used to convey sensed UV radiation to the UV sensor(s) 114.

A visible light sensor 116 is also mounted on the mobile cart 102, and each is also shielded from the UV radiation sources 106. The visible light sensors 116 are each operable to sense the intensity of the visible light reflected by the reflectors 112. The visible light sensors 116 are also operable to supply (via either wired or wireless techniques) a visible light intensity signal representative of the sensed intensity to the processor 118. The visible light sensors 116 may be implemented using any one of numerous known visible light sensors now known or developed in the future. Some non-limiting examples of suitable visible light sensors 116 include various photoresistor sensors, photodiode sensors, phototransistor sensors, just to name a few. In the depicted embodiment, a visible light sensor 116 is mounted on each arm 104. In other embodiments, one or more light sensors 116 may be mounted on the mobile cart 102, remote from each arm 104. In this embodiment, optical fibers may be used to convey sensed visible light to the visible light sensor(s) 116.

The processor 118 is coupled to receive the UV intensity signals from each UV sensor 114 and the visible light intensity signals from each visible light sensor 116. The processor 118 is configured (e.g., suitably programmed) to determine the intensity of the UV radiation on the surfaces 122 based on the UV intensity signals and the visible light intensity signals. Although the processor 118 may be configured to determine the UV radiation intensity on the surfaces 122 using any one of numerous techniques, in one embodiment, the processor determines the ratio of the UV intensity signal and the visible light intensity signal, and determines the intensity of the UV radiation on the surface from this determined ratio.

Additionally, because dose is proportional to intensity, the processor 118 is also configured to determine the UV dose to the surfaces 122 based at least in part on the determined intensity of the UV radiation. More specifically, and as is generally known, dose (D) is equal to the product of intensity (I) and time (t) (e.g., D=I×t). To determine the dose, the processor 118 can, in one embodiment, be configured to measure the time of the UV intensity signals associated with each reflector 112. In another embodiment, the processor 118 may use the speed of the mobile cart 102, and the movement distance of the mobile cart 102, to convert intensity to dose. In such an embodiment, the system additionally includes a speedometer 124. The speedometer 124 is mounted on, and is configured to sense the speed of, the mobile cart 124, and to supply a speed signal representative thereof to the processor 118. The processor 118 is further configured, upon receipt of the speed signal, to determine the UV dose to the surface based additionally on the speed signal.

Before proceeding further, it is noted that in most instances, the aircraft cabin will also be illuminated with conventional aircraft lighting, which is also visible light. Thus, it may be desirable to distinguish the conventional aircraft light from the visible light emitted by the visible light source. In one embodiment, this is accomplished by pulsing the visible light source 108 on and off, and the difference between the on and off pulses from the visible light sensors 116 may be used as the visible light intensity signals. With this embodiment, the processor 118 is preferably further configured to pulse the visible light source on and off, and to do so at a predetermined frequency. In an alternative embodiment, the visible light source 108 may be chosen to be a specific color, and the visible light sensors 116 may be equipped with color filters so that each detects only the color emitted by the visible light source 108. This will allow the visible light sensors 116 to only be sensitive to the light from the visible light source 108.

It should additionally be noted that the processor 118 generally represents the hardware, circuitry, processing logic, and/or other components configured to facilitate communications and/or interaction between the elements of the sanitization system 100 and perform additional processes, tasks and/or functions to support operation of the sanitization system 100, as described herein. Depending on the embodiment, the processor 118 may be implemented or realized with a general purpose processor, a controller, a microprocessor, a microcontroller, a content addressable memory, a digital signal processor, an application specific integrated circuit, a field programmable gate array, any suitable programmable logic device, discrete gate or transistor logic, processing core, discrete hardware components, or any combination thereof, designed to perform the functions described herein. In practice, the processing system 108 includes processing logic that may be configured to carry out the functions, techniques, and processing tasks associated with the operation of the aircraft system 100 described in greater detail below. Furthermore, the steps of a method or algorithm described in connection with the embodiments disclosed herein may be embodied directly in hardware, in firmware, in a software module executed by the processor 108, or in any practical combination thereof. In accordance with one or more embodiments, the processing system 108 includes or otherwise accesses a data storage element, such as a memory (e.g., RAM memory, ROM memory, flash memory, registers, a hard disk, or the like) or another suitable non-transitory short or long term storage media capable of storing computer-executable programming instructions or other data for execution that, when read and executed by the processing system 108, cause the processing system 108 to execute and perform one or more of the processes, tasks, operations, and/or functions described herein.

Returning now to the description, and as FIGS. 1 and 2 depict, the system 100 may also include a wireless transmitter 126 and a receiver 128. The wireless transmitter 126 is in operable communication with the processor 118 and is responsive to commands from the processor 118 to wirelessly transmit at least data, to the receiver 128, that is representative of the UV dose to the surface 122. Although the transmitter 126 is depicted as a separate functional block, it will be appreciated that the transmitter 126 may be integrally formed as part of the processor 118 or be implemented as a device that is separate from the processor 118.

The receiver 128 is in operable communication with, and receives the data transmitted from, the wireless transmitter 126. In a particular preferred embodiment, the receiver 128 is disposed within a hand-held device 132, such as a smartphone, a tablet computer, or any one of numerous other hand-held devices. The hand-held device 132 includes at least a display 134, such as a touchscreen display, that provides real-time indication of at least the UV dose for each surface 122 on which a reflector 112 is disposed.

In some embodiments, the system 100 may also include a memory 136 that has stored therein a library of disinfectant-related constants for various bacteria, viruses, or other microorganisms. The memory 136 may be disposed on the mobile cart 102, the hand-held device 132, or both. The processor 118 (and/or hand-held device 132), using the stored constants, may additionally be configured to determine, based at least in part on the UV dose to the surface, an amount of disinfection of one or more substances (e.g., bacteria, viruses, microorganisms) from the surfaces 122. For example, the processor 118 and/or hand-held device 132 can estimate the percentage disinfection for each substance. It will be appreciated that the memory 136 may also have stored therein surface-specific constants, that may be used by the processor 118 and/or hand-held device 132, to more accurately estimate the percentage disinfection of particular types of surfaces 122.

In some embodiments, the mobile cart 102 may also be equipped with a user interface, such as a cart-mounted display device 138 that is in operable communication with the processor 118. Thus, in addition to transmitting data to the hand-held device 132, the processor 118 may also command the display device 138 to render information for use by the operator of the mobile cart 102. For example, if the determined UV intensity is not at a desired level, the processor 118 can command the display device 138 to render an alert to the operator to speed up or slow down movement of the mobile cart 102 through the aisle 302 to ensure adequate disinfection.

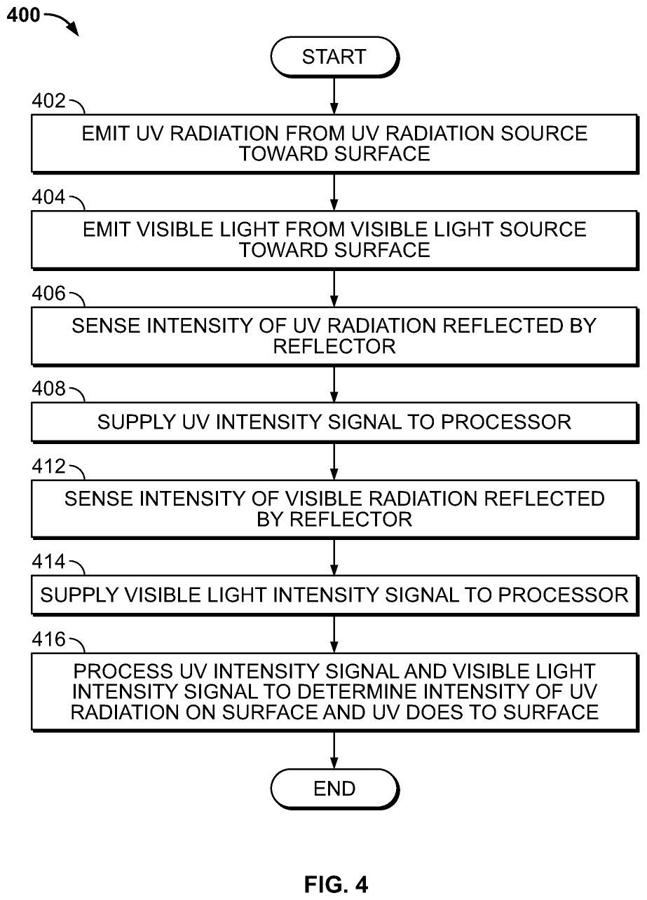

Having described the overall structure the system 100, and the functions of various components, devices, and sub-systems that comprise the system 100, an embodiment of a method of sanitizing a surface within an aircraft using the system 100 will now be briefly described. In doing so, reference should now be made to FIG. 4, which depicts the described methodology in flowchart form. It should be noted that although the method is depicted and described in a particular order, some steps of the process need not be performed in the depicted or described order. Moreover, while the method is described for a single surface 122, it will be appreciated that it applicable to multiple surfaces.

The depicted method 400 begins by emitting UV radiation from the UV radiation source 106 toward the surface 122 (402) and emitting visible light is emitted from the visible light source 108 toward the surface (404). The UV sensor 114 is used to sense the intensity of the UV radiation that is reflected by a reflector 112 that is disposed on on at least a portion of a surface 122 (406), and a UV intensity signal is supplied from the UV sensor 114 to the processor 118 (408). The visible light sensor 116 is used to sense the intensity of the visible light reflected by the reflector 112 (412), and a visible light intensity signal is supplied from the visible light sensor 116 to the processor 118 (414). The processor 118 processes the UV intensity signal and the visible light intensity signal to determine at least the intensity of the UV radiation on the surface 122 and a UV dose to the surface 122 (416).

The system and method described herein uses UV radiation to sanitize aircraft cabin surfaces and provides real-time feedback as to whether the sanitization has been done correctly and thoroughly.

Those of skill in the art will appreciate that the various illustrative logical blocks, modules, circuits, and algorithm steps described in connection with the embodiments disclosed herein may be implemented as electronic hardware, computer software, or combinations of both. Some of the embodiments and implementations are described above in terms of functional and/or logical block components (or modules) and various processing steps. However, it should be appreciated that such block components (or modules) may be realized by any number of hardware, software, and/or firmware components configured to perform the specified functions. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention. For example, an embodiment of a system or a component may employ various integrated circuit components, e.g., memory elements, digital signal processing elements, logic elements, look-up tables, or the like, which may carry out a variety of functions under the control of one or more microprocessors or other control devices. In addition, those skilled in the art will appreciate that embodiments described herein are merely exemplary implementations.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC.

Techniques and technologies may be described herein in terms of functional and/or logical block components, and with reference to symbolic representations of operations, processing tasks, and functions that may be performed by various computing components or devices. Such operations, tasks, and functions are sometimes referred to as being computer-executed, computerized, software-implemented, or computer-implemented. In practice, one or more processor devices can carry out the described operations, tasks, and functions by manipulating electrical signals representing data bits at memory locations in the system memory, as well as other processing of signals. The memory locations where data bits are maintained are physical locations that have particular electrical, magnetic, optical, or organic properties corresponding to the data bits. It should be appreciated that the various block components shown in the figures may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices.

When implemented in software or firmware, various elements of the systems described herein are essentially the code segments or instructions that perform the various tasks. The program or code segments can be stored in a processor-readable medium or transmitted by a computer data signal embodied in a carrier wave over a transmission medium or communication path. The “computer-readable medium”, “processor-readable medium”, or “machine-readable medium” may include any medium that can store or transfer information. Examples of the processor-readable medium include an electronic circuit, a semiconductor memory device, a ROM, a flash memory, an erasable ROM (EROM), a floppy diskette, a CD-ROM, an optical disk, a hard disk, a fiber optic medium, a radio frequency (RF) link, or the like. The computer data signal may include any signal that can propagate over a transmission medium such as electronic network channels, optical fibers, air, electromagnetic paths, or RF links. The code segments may be downloaded via computer networks such as the Internet, an intranet, a LAN, or the like.

Some of the functional units described in this specification have been referred to as “modules” in order to more particularly emphasize their implementation independence. For example, functionality referred to herein as a module may be implemented wholly, or partially, as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices, or the like. Modules may also be implemented in software for execution by various types of processors. An identified module of executable code may, for instance, comprise one or more physical or logical modules of computer instructions that may, for instance, be organized as an object, procedure, or function. Nevertheless, the executables of an identified module need not be physically located together, but may comprise disparate instructions stored in different locations that, when joined logically together, comprise the module and achieve the stated purpose for the module. Indeed, a module of executable code may be a single instruction, or many instructions, and may even be distributed over several different code segments, among different programs, and across several memory devices. Similarly, operational data may be embodied in any suitable form and organized within any suitable type of data structure. The operational data may be collected as a single data set, or may be distributed over different locations including over different storage devices, and may exist, at least partially, merely as electronic signals on a system or network.

In this document, relational terms such as first and second, and the like may be used solely to distinguish one entity or action from another entity or action without necessarily requiring or implying any actual such relationship or order between such entities or actions. Numerical ordinals such as “first,” “second,” “third,” etc. simply denote different singles of a plurality and do not imply any order or sequence unless specifically defined by the claim language. The sequence of the text in any of the claims does not imply that process steps must be performed in a temporal or logical order according to such sequence unless it is specifically defined by the language of the claim. The process steps may be interchanged in any order without departing from the scope of the invention as long as such an interchange does not contradict the claim language and is not logically nonsensical.

Furthermore, depending on the context, words such as “connect” or “coupled to” used in describing a relationship between different elements do not imply that a direct physical connection must be made between these elements. For example, two elements may be connected to each other physically, electronically, logically, or in any other manner, through one or more additional elements.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A portable sanitization system for sanitizing a surface in an aircraft, comprising:
a mobile cart movable within the aircraft;
an arm coupled to, and movable relative to, the mobile cart;
an ultraviolet (UV) radiation source mounted on the arm and operable to emit UV radiation toward the surface;
a visible light source mounted on the arm and operable to emit visible light toward the surface;
a reflector disposed on at least a portion of the surface and configured to reflect at least a portion of the UV radiation and at least a portion of the visible light emitted toward the surface;
a UV sensor mounted on the mobile cart and shielded from the UV radiation source, the UV sensor operable to (i) sense an intensity of the UV radiation reflected by the reflector and (ii) supply a UV intensity signal representative thereof;
a visible light sensor mounted on the mobile cart and shielded from the UV radiation source, the visible light sensor operable to (i) sense an intensity of the visible light reflected by the reflector and (ii) supply a visible light intensity signal representative thereof; and
a processor coupled to receive the UV intensity signal from the UV sensor and the visible light intensity signal from the visible light sensor, the processor configured to determine (i) the intensity of the UV radiation on the surface based on the UV intensity signal and the visible light intensity signal and (ii) a UV dose to the surface based at least in part on the determined intensity of the UV radiation.

2. The portable sanitization system of claim 1, further comprising:
a speedometer mounted on the mobile cart, the speedometer configured to sense a speed of the mobile cart and supply a speed signal representative thereof,
wherein the processor is further coupled to receive the speed signal from the speedometer and is further configured to determine the UV dose to the surface based additionally on the speed signal.

3. The portable sanitization system of claim 1, further comprising:
a wireless transmitter in operable communication with the processor, the wireless transmitter responsive to commands from the processor to wirelessly transmit at least data representative of the UV dose to the surface; and
a receiver in operable communication with the wireless transmitter to receive the data transmitted therefrom.

4. The portable sanitization system of claim 3, wherein the receiver is disposed within a hand-held device.

5. The portable sanitization system of claim 1, wherein the processor is further configured to determine, based at least in part on the UV dose to the surface, an amount of disinfection of one or more substances from the surface.

6. The portable sanitization system of claim 1, wherein the reflector is formed integrally with the surface.

7. The portable sanitization system of claim 1, wherein the reflector is readily disposable on and removable from the surface.

8. The portable sanitization system of claim 1, wherein the visible light source comprises a light emitting diode (LED).

9. The portable sanitization system of claim 1, wherein the processor is further configured to pulse the visible light source on and off at a predetermined frequency.

10. The portable sanitization system of claim 1, wherein the processor is configured to:
determine a ratio of the UV intensity signal and the visible light intensity signal; and
determine the intensity of the UV radiation on the surface from the determined ratio.

11. A portable sanitization system for sanitizing surfaces in an aircraft, comprising:
a mobile cart movable within the aircraft;
a plurality of arms coupled to, and extendable from, the mobile cart;
a plurality of ultraviolet (UV) radiation sources, each UV radiation source mounted on a different one of the arms and operable to emit UV radiation toward the surfaces;
a visible light source mounted on the arm and operable to emit visible light toward the surfaces;
a plurality of reflectors, each reflector disposed on at least a portion of a different one of the surfaces and configured to reflect at least a portion of the UV radiation and at least a portion of the visible light emitted toward the surfaces;
a plurality of UV sensors, each UV sensor mounted on a different one of the arms and shielded from the UV radiation sources, each UV sensor operable to (i) sense an intensity of the UV radiation reflected by each reflector and (ii) supply a UV intensity signal representative thereof;
a plurality of visible light sensors, each visible light sensor mounted on a different one of the arms and shielded from the UV radiation source, each visible light sensor operable to (i) sense an intensity of the visible light reflected by each reflector and (ii) supply a visible light intensity signal representative thereof;
a processor coupled to receive the UV intensity signals from the UV sensors and the visible light intensity signals from the visible light sensors, the processor configured to (i) pulse each visible light source on and off at a predetermined frequency, (ii) determine the intensity of the UV radiation on each surface based on the UV intensity signals and the visible light intensity signals, and (iii) determine a UV dose to each surface based at least in part on the determined intensity of the UV radiation;
a wireless transmitter in operable communication with the processor, the wireless transmitter responsive to commands from the processor to wirelessly transmit at least data representative of the UV dose to each surface; and
a receiver in operable communication with the wireless transmitter to receive the data transmitted therefrom.

12. The portable sanitization system of claim 11, further comprising:
a speedometer mounted on the mobile cart, the speedometer configured to sense a speed of the mobile cart and supply a speed signal representative thereof,
wherein the processor is further coupled to receive the speed signal from the speedometer and is further configured to determine the UV dose to the surface based additionally on the speed signal.

13. The portable sanitization system of claim 11, wherein the receiver is disposed within a hand-held device.

14. The portable sanitization system of claim 11, wherein the processor is further configured to determine, based at least in part on the UV dose to each surface, an amount of disinfection of one or more substances from each surface.

15. The portable sanitization system of claim 11, wherein each reflector is formed integrally with a different one of the surfaces.

16. The portable sanitization system of claim 11, wherein each reflector is readily disposable on and removable from a different one of the surfaces.

17. The portable sanitization system of claim 11, wherein the visible light source comprises a light emitting diode (LED).

18. The portable sanitization system of claim 11, wherein the processor is configured to:
determine a ratio of the UV intensity signals and the visible light intensity signals; and
determine the intensity of the UV radiation on each surface from the determined ratio.

19. A method of sanitizing a surface within an aircraft using a mobile cart within the aircraft, the mobile cart having an arm that is coupled thereto and that is movable relative thereto, the arm having an ultraviolet (UV) radiation source and a visible light source mounted thereon, and a UV sensor and a visible light source mounted on the mobile cart, the method comprising the steps of:
emitting UV radiation from the UV radiation source toward the surface;
emitting visible light from the visible light source toward the surface;
sensing, using the UV sensor, an intensity of the UV radiation reflected by a reflector that is disposed on a least a portion of the surface;
supplying a UV intensity signal from the UV sensor to a processor, the UV intensity signal representative of the sensed intensity of the UV radiation reflected by the reflector;
sensing, using the visible light sensor, an intensity of the visible light reflected by the reflector;
supplying a visible light intensity signal from the visible light sensor to the processor, the visible light intensity signal representative of the sensed intensity of the visible light reflected by the reflector; and
processing, in the processor, the UV intensity signal and the visible light intensity signal to determine the intensity of the UV radiation on the surface.

20. The method of claim 19, further comprising:
processing, in the processor, the UV intensity signal and the visible light intensity signal to determine a UV dose to the surface; and
wirelessly transmitting the determined intensity of the UV radiation and the determined UV dose to a remote receiver.
